(19) United States
(12) Patent Application Publication
(10) Pub. No.: US 2024/0238448 A1
(43) Pub. Date: Jul. 18, 2024

(54) USE OF CHEMICAL EPIGENETIC MODIFIERS TO MODULATE GENE EXPRESSION FROM VECTORS

(71) Applicant: The University of North Carolina at Chapel Hill, Chapel Hill, NC (US)

(72) Inventors: Nathaniel Hathaway, Chapel Hill, NC (US); Matthew Hirsch, Chapel Hill, NC (US); Jessica Umana, Durham, NC (US); Liujiang Song, Chapel Hill, NC (US)

(21) Appl. No.: 18/558,886

(22) PCT Filed: May 6, 2022
(86) PCT No.: PCT/US2022/028011

§ 371 Date: Nov. 3, 2023

Related U.S. Application Data
(60) Provisional application No. 63/185,013, filed on May 6, 2021.

(51) Int. Cl. A61K 48/00; A61K 38/12; C12N 15/62; C12N 15/85

(52) U.S. Cl. CPC A61K 48/0066; A61K 38/12; C12N 15/62; C12N 15/85; C07K 2319/80

(57) ABSTRACT

This invention relates to methods and compositions for gene therapy. In particular, the invention relates to methods and compositions for modulating transgene expression from transgene delivery vectors by recruiting epigenetic modifiers to the vector. Using these methods, transgene delivery vectors can be more precisely regulated to produce increased amounts of the transgene product when needed and to decrease expression when needed, thereby providing maximum benefits for gene therapy while minimizing toxicity.

### STATEMENT OF PRIORITY

This application claims the benefit of U.S. Provisional Application Ser. No. 63/185,013, filed May 6, 2021, the entire contents of which are incorporated by reference herein.

### GOVERNMENT SUPPORT

This invention was made with government support under Grant Number GM118653 awarded by the National Institutes of Health. The government has certain rights in the invention.

### FIELD OF THE INVENTION

This invention relates to methods and compositions for gene therapy. In particular, the invention relates to methods and compositions for modulating transgene expression from transgene delivery vectors by recruiting epigenetic modifiers to the vector. Using these methods, transgene delivery vectors can be more precisely regulated to produce increased amounts of the transgene product when needed and to decrease expression when needed, thereby providing maximum benefits for gene therapy while minimizing toxicity.

### BACKGROUND OF THE INVENTION

Adeno-Associated virus (AAV) gene therapy applications for the treatment of diverse genetic diseases have been applied in >1,000 humans to date with optimistic therapeutic results observed for broad disorders of the muscle, blood, brain, and those affecting vision (Li et al., Nat. Rev. Genet. 21(4):255 (2020)). In all clinical studies thus far, the AAV vectors administered have one thing in common: they remain uncontrolled at the level of transgene expression. Fortunately, minimal to no deleterious consequences of constitutive transgene production have been reported to date; however, the allure of precise and specific modulation of vector control to address transgene product safety concerns remains in all gene therapy applications, including those of viral and non-viral delivery formats.

The present invention overcomes shortcomings in the art by providing novel methods and compositions for modulating transgene expression from transgene delivery vectors.

### SUMMARY OF THE INVENTION

The present invention is based on the discovery that transgene expression from vectors can be modulated by recruiting epigenetic modifiers to the vector to increase or decrease transgene expression. Using these methods, transgene delivery vectors can be more precisely regulated to produce increased amounts of the transgene product when needed and to decrease expression when needed, thereby providing maximum benefits for gene therapy while minimizing toxicity.

Previous work by the inventors has demonstrated the specific activation and repression of chromosomal gene expression via the recruitment of modifiers that generate euchromatin or heterochromatin, respectively (Hathaway et al., Cell 149(7): 1447 (2012); Vignaux et al., PLoS One 14(7):e0217699 (2019)). This strategy is reliant on a fusion protein consisting of a targeted zinc finger (ZF) DNA binding domain to a host domain, FKBP, that interacts with bi-functional Chemical Epigenetic Modifiers (CEMs) (Butler et al., ACS Synth. Biol. 7(1):38 (2018)). Following reports using this system in specific chromosomal gene activation or repression (Chiarella et al., J. Vis. Exp. 2018(139):58222; Gryder et al., Nat. Genet. 51(12):1714 (2019); Chiarella et al., Nat. Biotechnol. 38(1):50 (2020)), extrapolated proof-of-concept studies regulating transduced AAV vector episomes have generated data in human cells demonstrating the ability to control AAV transgene expression via recruitment of chromatin modifiers. Specifically, individual recruitment of BRD4 to AAV episomes resulted in the rapid and significant enhancement of transgene expression. These novel observations in well controlled and rigorous experiments demonstrate that AAV episomes are naturally restricted for expression in human cells and allude to the ability to specifically induce therapeutic transgene expression at a fixed AAV vector dose.

Thus, one aspect of the invention relates to methods of modulating expression of a transgene from a transgene delivery vector, the method comprising:
providing a transgene delivery vector comprising a polynucleotide comprising a transgene expression cassette and a nucleic acid binding domain recognition sequence;
contacting the transgene delivery vector with a fusion protein comprising a nucleic acid binding domain that binds to the recognition sequence fused to a domain that binds a chemical epigenetic modifier; and
contacting the transgene delivery vector with the chemical epigenetic modifier; thereby modulating expression of the transgene from the transgene delivery vector.

Another aspect of the invention relates to methods of modulating expression of a transgene from a transgene delivery vector in a subject, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a chemical epigenetic modifier; and
administering to the subject the chemical epigenetic modifier; thereby modulating expression of the transgene.

A further aspect of the invention relates to methods of treating a disorder that is treatable by expression of a transgene from a transgene delivery vector in a subject in need thereof, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a chemical epigenetic modifier; and
administering to the subject the chemical epigenetic modifier;
thereby treating the disorder.

Another aspect of the invention relates to transgene delivery vectors comprising a polynucleotide comprising a DNA binding domain recognition sequence and a transgene expression cassette comprising a transgene, and cells, compositions, and kits comprising the same.

These and other aspects of the invention are set forth in more detail in the description of the invention below.

### BRIEF DESCRIPTION OF THE DRAWINGS

FIG. 1 shows the general structure of CEMs.

FIG. 2 shows the structure of activating and repressive CEMs.

FIG. 3 shows the structure and use of zinc finger proteins.

FIG. 4 shows an example of the structure for ZF-CEM technology.

FIG. 5 shows control of AAV transgene with CEM. An AAV construct expressing a luciferase reporter with zinc finger (ZF) binding domains inserted upstream of a gene of interest (GOI) is modulated by a ZF-FKBP targeting protein where a small molecule (CEM87) recruits endogenous BRD4 epigenetic activator to regulate GOI expression.

FIG. 6 shows the effect of the ZF-CEM method.

FIG. 7 shows the effect of the ZF-CEM method using an AAV vector that expresses the fusion protein.

FIG. 8 shows the effect of the ZF-CEM method on different AAV serotypes.

FIG. 9 shows the effect of the ZF-CEM method 24 h and 48 h after CEM treatment.

FIG. 10 shows chemical activation of AAV vectors in human cells. AAV2-GFP vectors containing a Zinc Finger (ZF) binding domain (BD) were evaluated in 293 cells+/−a ZF-FKBP fusion protein responsive to CEM. CEM addition increased GFP specifically from vectors harboring the ZF binding domain in a dose-dependent manner.

FIG. 11 shows CEM87 pharmacokinetics (PK). Mice were administered a single dose of CEM87 intravenously (IV), and intraperitoneally (IP), and PK estimates were calculated.

FIG. 12 shows AAV-CEMtrol constructs.

FIG. 13 shows a study timeline for in vivo effects.

FIG. 14 shows a summary of ZF-CEM technology.

FIG. 15 shows ZF-CEM technology exhibits dose dependent chemical control with CEM87 at both low and high viral genomes per cell. At a higher viral genomes per cell dose of virus, higher fold change in transgene (firefly luciferase) expression is seen.

FIG. 16 shows ZF-CEM technology (20K viral genomes per cell) is able to increase the levels of transgene (firefly luciferase) expression in a statistically significant manner as early as 24 hours and continues to have effects up to 96 hours post CEM87 addition. The highest fold change in luciferase expression is seen 24 hours after CEM87 dosage (48 hours after AAV infection).

FIG. 17 shows the impact of ZF-CEM technology (20K viral genomes per cell) on transgene expression is reversible through washout or washout with the addition of FK506, a chemical capable of competing off CEM87 from the transgene it is recruited to. Reversibility is achieved within 24 hours of removal of CEM87 through washout or washout and treatment with 100× more FK506 (20 μM FK506).

FIG. 18 shows CEM114 can be used in conjunction with ZF-CEM technology (20K viral genomes per cell) to increase AAV transgene expression.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, N Y, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### General Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together.

The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “tropism” as used herein refers to preferential but not necessarily exclusive entry of the vector (e.g., virus vector) into certain cell or tissue type(s) and/or preferential but not necessarily exclusive interaction with the cell surface that facilitates entry into certain cell or tissue types, optionally and preferably followed by expression (e.g., transcription and, optionally, translation) of sequences carried by the vector contents (e.g., viral genome) in the cell, e.g., for a recombinant virus, expression of the heterologous nucleotide sequence(s).

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g., U.S. Pat. No. 9,636,370 Mccown et al., and US patent publication 2017/0360960 Gray et al.). Vectors (e.g., virus vectors, e.g., AAV capsids) expressing specific tropism profiles may be referred to as “tropic” for their tropism profile, e.g., neuro-tropic, liver-tropic, etc.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides) but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a virus vector, it is meant that the virus vector is at least partially separated from at least some of the other components in the starting material.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease and encompass any type of prophylactic treatment to reduce the incidence of the condition or delay the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “heterologous nucleotide sequence” or “heterologous nucleic acid,” with respect to a virus or other vector, is a sequence or nucleic acid, respectively, that is not naturally occurring in the virus or other vector. Generally, the heterologous nucleic acid or nucleotide sequence comprises an open reading frame that encodes a polypeptide and/or a nontranslated RNA.

A “vector” refers to a compound used as a vehicle to carry foreign genetic material into another cell, where it can be replicated and/or expressed. A vector containing foreign or heterologous nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to as transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “viral vector” and “delivery vector” (and similar terms) in a specific embodiment generally refers to a virus particle that functions as a nucleic acid delivery vehicle, and which comprises the viral nucleic acid (i.e., the vector genome) packaged within the virion. Viral vectors according to the present invention may include chimeric AAV capsids according to the invention and can package an AAV or rAAV genome or any other nucleic acid including viral nucleic acids. Alternatively, in some contexts, the terms “viral vector” and “delivery vector” (and similar terms) may be used to refer to the vector genome (e.g., vDNA) in the absence of the virion and/or to a viral capsid that acts as a transporter to deliver molecules tethered to the capsid or packaged within the capsid.

The term “template” or “substrate” is used herein to refer to a polynucleotide sequence that may be replicated to produce the viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, the term “amino acid” encompasses any naturally occurring amino acids, modified forms thereof, and synthetic amino acids, including non-naturally occurring amino acids.

Naturally occurring, levorotatory (L-) amino acids are shown in Table 1.

TABLE 1

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 2) or can be an amino acid that is modified by post-translation modification (e.g., acetylation, amidation, formylation, hydroxylation, methylation, phosphorylation or sulfatation).

TABLE 2
Amino Acid Residue Derivatives

| Modified Amino Acid Residue | Abbreviation |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid as described by Wang et al., (2006) Annu. Rev. Biophys. Biomol. Struct. 35:225-49.

A “functional fragment” of a polypeptide or protein, as used herein, means a portion of a larger polypeptide that substantially retains its biological activity.

As used herein, the term “derivative” is used to refer to a polypeptide which differs from a naturally occurring protein or a functional fragment by minor modifications to the naturally occurring polypeptide, but which substantially retains the biological activity of the naturally occurring protein. Minor modifications include, without limitation, changes in one or a few amino acid side chains, changes to one or a few amino acids (including deletions, insertions, and/or substitutions) (e.g., less than about 20, 15, 10, 9, 8, 7, 6, 5, 4, 3, or 2 changes), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol.

The term “substantially retains,” as used herein, refers to a fragment, derivative, or other variant of a polypeptide that retains at least about 50% of the activity of the naturally occurring polypeptide (e.g., binding to recognition sequence), e.g., about 60%, 70%, 80%, 90% or more.

### Methods of Modulating Transgene Expression

One aspect of the invention relates to methods of modulating expression of a transgene from a transgene delivery vector, the method comprising:
providing a transgene delivery vector comprising a polynucleotide comprising a transgene expression cassette and a nucleic acid binding domain recognition sequence;
contacting the transgene delivery vector with a fusion protein comprising a nucleic acid binding domain that binds to the recognition sequence fused to a domain that binds a chemical epigenetic modifier; and
contacting the transgene delivery vector with the chemical epigenetic modifier; thereby modulating expression of the transgene from the transgene delivery vector.

Another aspect of the invention relates to methods of modulating expression of a transgene from a transgene delivery vector in a subject, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a chemical epigenetic modifier; and
administering to the subject the chemical epigenetic modifier; thereby modulating expression of the transgene.

A further aspect of the invention relates to methods of treating a disorder that is treatable by expression of a transgene from a transgene delivery vector in a subject in need thereof, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a chemical epigenetic modifier; and
administering to the subject the chemical epigenetic modifier;
thereby treating the disorder.

The transgene delivery vector may be any type of vector known to be useful for delivering a polynucleotide to a cell. In some embodiments, the transgene delivery vector is a viral vector, e.g., a viral genome. Examples of viral vectors include, without limitation, an adeno-associated virus, retrovirus, lentivirus, poxvirus, alphavirus, baculovirus, vaccinia virus, herpes virus, Epstein-Barr virus, or adenovirus vector.

In some embodiments, the transgene delivery vector is a non-viral vector. Examples of non-viral vectors include, without limitation, a plasmid, liposome, electrically charged lipid, nucleic acid-protein complex, or biopolymer.

The transgene may encode any product for which delivery and expression is desired, as discussed further below. In some embodiments, the transgene encodes a protein. In other embodiments, the transgene encodes a functional nucleic acid, e.g., an antisense nucleic acid or an inhibitory RNA.

In some embodiments of the methods, the fusion protein is provided separately from the transgene delivery vector. For example, the transgene delivery vector and the fusion protein may be separately delivered into a cell or to a subject. In some embodiments, the fusion protein may already be in the cell, e.g., because the cell expresses the fusion protein.

In other embodiments, the polynucleotide further comprises a sequence encoding the fusion protein, e.g., so that the transgene-encoded product and the fusion protein are both produced by the transgene delivery vector. In these embodiments, the transgene and the sequence encoding the fusion protein may be operably linked to separate promoters (which may be the same promoter or different promoters) or may be operably linked to a single promoter, which may optionally be a bidirectional promoter. In other embodiments, the fusion protein may be expressed from the sequence encoding the fusion protein due to the inherent promoter activity of AAV ITRs.

The nucleic acid binding domain recognition sequence may be any nucleotide sequence that is specifically recognized and bound by a nucleic acid binding protein such that the presence of the nucleic acid binding domain recognition sequence in the transgene delivery vector recruits a fusion protein comprising the nucleic acid binding protein. The nucleic acid binding domain recognition sequence may comprise two or more sequences (e.g., 2, 3, 4, 5, 6, or sequences) that are binding sites for the nucleic acid binding protein such that two or more nucleic acid binding proteins bind to the transgene delivery vector. For example, the nucleic acid binding domain recognition sequence may comprise 6 binding sites for zinc finger proteins so that a fusion protein comprising 6 zinc finger proteins may bind the transgene delivery vector in a sequence specific manner. In other embodiments, the multiple binding sites may recruit multiple fusion proteins to amplify the expression modulation to levels greater than can be achieved by a single fusion protein.

The nucleic acid binding domain recognition sequence may be recognized by a protein (e.g., zinc finger proteins) or a nucleic acid (e.g., RNA guided Cas proteins). In some embodiments, the nucleic acid binding domain is a DNA binding domain that is specifically recognized and bound by a DNA binding protein. In some embodiments, the DNA binding domain comprises one of a zinc finger DNA binding domain, a helix-loop-helix DNA binding domain, a bZIP DNA binding domain, an HMG-box DNA binding domain, a transcription activator-like effector DNA binding domain, a transcription factor DNA binding domain, or a restriction endonuclease DNA binding domain. In particular embodiments, the DNA binding domain may be, without limitation, a domain from GAL4, LexA, GCN4, THY1, SYN1, NSE/RU5′, AGRP, CALB2, CAMK2A, CCK, CHAT, DLX6A, EMX1, Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu196, or TALES.

In some embodiments, the nucleic acid binding domain is a RNA binding domain. In particular embodiments, the RNA binding domain may be, without limitation, a domain that binds a MS2, PP7, GA, or Qβ hairpin motif.

The portion of the fusion protein that is a domain that binds a CEM may be any polypeptide that specifically recognizes and binds to a portion of the CEM that is a ligand for the domain. In some embodiments, the domain that binds a CEM is FK506 binding protein and the CEM comprises FK506. In other embodiments, the domain that binds a CEM is a mutant FK506 binding protein (e.g., F6V) that binds a bumped CEM ligand (see, e.g., Lu et al., ACS Synth. Biol. 11:1397 (2022), incorporated by reference herein in its entirety).

CEMs useful in the methods of the invention may be any bifunctional small molecule (e.g., less than 1500 Da) that is capable of binding the fusion protein of the invention and a chromatin modifier. Useful CEMs include, without limitation, those described in WO 2019/028426, incorporated herein by reference in its entirety.

In some embodiments, modulating expression of a transgene comprises increasing expression of the transgene and contacting the transgene delivery vector with the CEM or administering the CEM to the subject increases expression of the transgene. In certain embodiments, the CEM binds to a transcriptional activator protein or complex that when recruited to the transgene delivery vector increases expression of the transgene. Examples of a transcriptional activator protein or complex include, without limitation, BRD4 or CBP/p300.

An exemplary CEM that binds FK506 binding protein and BRD4 is compound 1 (CEM87).

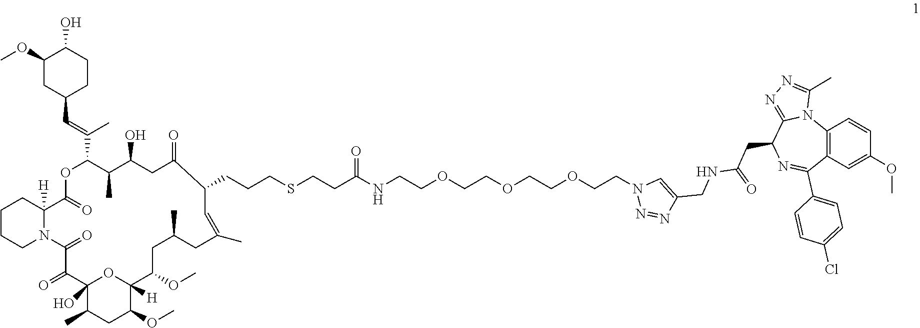

or a pharmaceutically acceptable salt thereof.

An exemplary CEM that binds FK506 binding protein and CBP/p300 is compound 2 (CEM114).

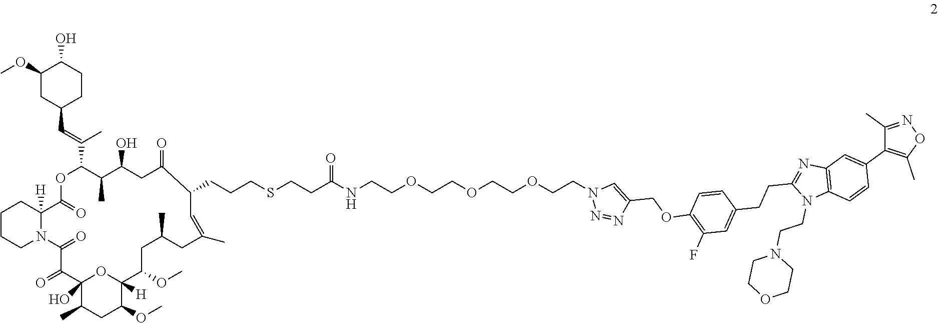 [SMILES: CO[C@H]1C[C@@H](C)C/C(C)=C/[C@@H](CCCSCCC(=O)NCCOCCOCCOCCn2cc(COc3ccc(CCc4nc5cc(-c6c(C)noc6C)ccc5n4CCN4CCOCC4)cc3F)nn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

or a pharmaceutically acceptable salt thereof.

In some embodiments, the methods may further comprise a subsequent step of blocking the increased expression of the transgene by contacting the transgene delivery vector with or administering to the subject an agent that inhibits binding of the fusion protein to the CEM. In this manner, overexpression of the transgene may be avoided. The agent that inhibits binding of the fusion protein to the CEM may be any agent that interferes with fusion protein: CEM binding. The agent may be the ligand that is the portion of the CEM recognized by the fusion protein. For example, when the fusion protein comprises FKBP, an excess amount of free FK506 may be provided which would bind FKBP and interfere with binding of the CEM to FKBP.

In other embodiments, the methods may further comprise a subsequent step of blocking the increased expression of the transgene by stopping the contacting the transgene delivery vector with or the administering to the subject the CEM. By strategically administering the CEM and stopping administration of the CEM, transgene expression may be modulated to keep expression at the desired level, e.g., high enough to have a therapeutic effect but not so high as to cause toxicity or side effects. Similarly, the amount of CEM administered can be modified to modulate transgene expression levels. Steps of increasing and blocking increased transgene expression levels may be repeated more than once, e.g., 2, 3, 4, 5, 6, 7, 8, 9, or 10 times or more, e.g., to maintain transgene expression at a desired level and/or for a desired length of time.

In some embodiments, modulating expression of a transgene comprises decreasing expression of the transgene and contacting the transgene delivery vector with the CEM or administering the CEM to the subject decreases expression of the transgene. In certain embodiments, the CEM binds to a transcriptional inhibitor protein or complex. Examples of a transcriptional inhibitor protein or complex include, without limitation, a histone deacetylase.

An exemplary CEM that binds FK506 binding protein and a histone deacetylase is compound 3 (CEM23).

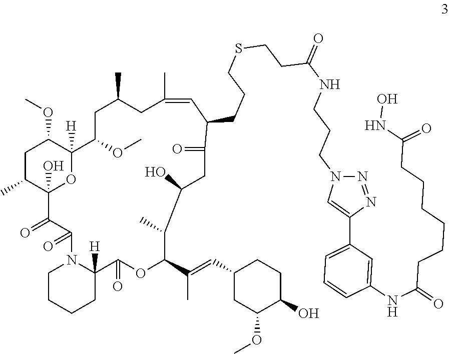 [SMILES: CO[C@H]1C[C@@H](C)C/C(C)=C\[C@@H](CCCSCCC(=O)NCCCn2cc(-c3cccc(NC(=O)CCCCCCC(=O)NO)c3)nn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]

or a pharmaceutically acceptable salt thereof.

In some embodiments, the methods may further comprise a subsequent step of blocking the decreased expression of the transgene by contacting the transgene delivery vector with or administering to the subject an agent that inhibits binding of the fusion protein to the CEM. In this manner, underexpression of the transgene may be avoided. The agent that inhibits binding of the fusion protein to the CEM may be any agent that interferes with fusion protein:CEM binding. The agent may be the ligand that is the portion of the CEM recognized by the fusion protein. For example, when the fusion protein comprises FKBP, an excess amount of free FK506 may be provided which would bind FKBP and interfere with binding of the CEM to FKBP.

In other embodiments, the methods may further comprise a subsequent step of blocking the decreased expression of the transgene by stopping the contacting the transgene delivery vector with or the administering to the subject the CEM. By strategically administering the CEM and stopping administration of the CEM, transgene expression may be modulated to keep expression at the desired level, e.g., high enough to have a therapeutic effect but not so high as to cause toxicity or side effects. Similarly, the amount of CEM administered can be modified to modulate transgene expression levels. Steps of decreasing and blocking decreased transgene expression levels may be repeated more than once, e.g., 2, 3, 4, 5, 6, 7, 8, 9, or 10 times or more, e.g., to maintain transgene expression at a desired level and/or for a desired length of time.

### Transgene Delivery Vectors

One aspect of the invention relates to a transgene delivery vector comprising a polynucleotide comprising a nucleic acid binding domain recognition sequence and a transgene expression cassette comprising a transgene.

As used herein, the term “transgene delivery vector” refers to a vector capable of delivering a transgene to a cell or to a subject and expressing the transgene in the cell or subject. The transgene delivery vector may be any type of vector known to be useful for delivering a polynucleotide to a cell. In some embodiments, the transgene delivery vector is a viral vector, e.g., a viral genome. Examples of viral vectors include, without limitation, an adeno-associated virus, retrovirus, lentivirus, poxvirus, alphavirus, baculovirus, vaccinia virus, herpes virus, Epstein-Barr virus, or adenovirus vector.

In some embodiments, the transgene delivery vector is a non-viral vector. Examples of non-viral vectors include, without limitation, a plasmid, liposome, electrically charged lipid, nucleic acid-protein complex, or biopolymer.

The transgene may encode any product for which delivery and expression is desired, as discussed further below. In some embodiments, the transgene encodes a protein. In other embodiments, the transgene encodes a functional nucleic acid, e.g., an antisense nucleic acid or an inhibitory RNA.

The nucleic acid binding domain recognition sequence serves as a binding site for a fusion protein comprising a nucleic acid binding domain that binds to the recognition sequence fused to a domain that binds a CEM. In some embodiments, the polynucleotide further comprises a sequence encoding the fusion protein, e.g., so that the transgene-encoded product and the fusion protein are both produced by the transgene delivery vector. In these embodiments, the transgene and the sequence encoding the fusion protein may be operably linked to separate promoters (which may be the same promoter or different promoters) or may be operably linked to a single promoter, which may optionally be a bidirectional promoter.

The nucleic acid binding domain recognition sequence may be any nucleotide sequence that is specifically recognized and bound by a nucleic acid binding protein such that the presence of the nucleic acid binding domain recognition sequence in the transgene delivery vector recruits a fusion protein comprising the nucleic acid binding protein. The nucleic acid binding domain recognition sequence may comprise two or more sequences (e.g., 2, 3, 4, 5, 6, or sequences) that are binding sites for the nucleic acid binding protein such that two or more nucleic acid binding proteins bind to the transgene delivery vector. For example, the nucleic acid binding domain recognition sequence may comprise 6 binding sites for zinc finger proteins so that a fusion protein comprising 6 zinc finger proteins may bind the transgene delivery vector in a sequence specific manner. In other embodiments, the multiple binding sites may recruit multiple fusion proteins to amplify the expression modulation to levels greater than can be achieved by a single fusion protein.

In some embodiments, the nucleic acid binding domain is a DNA binding domain that is specifically recognized and bound by a DNA binding protein. In some embodiments, the DNA binding domain comprises one of a zinc finger DNA binding domain, a helix-loop-helix DNA binding domain, a bZIP DNA binding domain, an HMG-box DNA binding domain, a transcription activator-like effector DNA binding domain, a transcription factor DNA binding domain, or a restriction endonuclease DNA binding domain. In particular embodiments, the DNA binding domain may be, without limitation, a domain from GAL4, LexA, GCN4, THY1, SYN1, NSE/RU5′, AGRP, CALB2, CAMK2A, CCK, CHAT, DLX6A, EMX1, Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu196, or TALES.

In some embodiments, the nucleic acid binding domain is a RNA binding domain. In particular embodiments, the RNA binding domain may be, without limitation, a domain that binds a MS2, PP7, GA, or Qβ hairpin motif.

The portion of the fusion protein that is a domain that binds a CEM may be any polypeptide that specifically recognizes and binds to a portion of the CEM that is a ligand for the domain. In some embodiments, the domain that binds a CEM is FK506 binding protein and the CEM comprises FK506.

Another aspect of the invention relates to a cell comprising the transgene delivery vector of the invention. The cell may be in vitro or in vivo.

A further aspect of the invention relates to a composition comprising the transgene delivery vector or the cell of the invention, e.g., a pharmaceutical composition comprising the transgene delivery vector or the cell of the invention and a pharmaceutically acceptable carrier. In some embodiments, the pharmaceutical composition further comprises the fusion protein and/or the CEM of the invention.

An additional aspect of the invention relates to a kit comprising the transgene delivery vector or the cell of the invention. In some embodiments, the kit further comprises the fusion protein and/or the CEM of the invention.

Another aspect of the invention relates to fusion proteins useful in the methods of the invention. In some embodiments, the fusion protein comprises FK506 binding protein or a mutant thereof (e.g., F36V) fused to a nucleic acid binding domain such as a zinc finger domain.

In some embodiments of the invention, the transgene delivery vector is a parvovirus vector. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

In some embodiments of the invention, the transgene delivery vector is a parvovirus within the genus Dependovirus. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 3. A number of additional AAV serotypes and clades have been identified (see, e.g., Gao et al., (2004) J. Virol. 78:6381-6388 and Table 3), which are also encompassed by the term “AAV.”

As discussed above, the parvovirus particles and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73: 939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-: 375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 3. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

The term “AAV viral vectors” includes “chimeric” AAV nucleic acid capsid coding sequence or AAV capsid protein is one that combines portions of two or more capsid sequences. A “chimeric” AAV virion or particle comprises a chimeric AAV capsid protein.

TABLE 3

| AAV | GenBank | AAV | GenBank | AAV | GenBank |
|---|---|---|---|---|---|
| Serotypes/Isolates | Accession Number | Serotypes/Isolates | Accession Number | Serotypes/Isolates | Accession Number |
| Clonal Isolates | | Hu S17 | AY695376 | Cy3 | AY243019 |
| Avian AAV ATCC VR-865 | AY186198, | Hu T88 | AY695375 | Cy5 | AY243017 |
| | AY629583, | | | | |
| | NC_004828 | | | | |
| Avian AAV strain DA-1 | NC_006263, | Hu T71 | AY695374 | Rh13 | AY243013 |
| | AY629583 | | | | |
| Bovine AAV | NC_005889, | Hu T70 | AY695373 | | |
| | AY388617 | | | | |
| AAV4 | NC_001829 | Hu T40 | AY695372 | Clade E | |
| AAV5 | AY18065, | Hu T32 | AY695371 | Rh38 | AY530558 |
| | AF085716 | | | | |
| Rh34 | AY243001 | Hu T17 | AY695370 | Hu66 | AY530626 |
| Rh33 | AY243002 | Hu LG15 | AY695377 | Hu42 | AY530605 |
| Rh32 | AY243003 | | | Hu67 | AY530627 |
| AAV10 | AY631965 | Clade C | | Hu40 | AY530603 |
| AAV11 | AY631966 | AAV 3 | NC_001729 | Hu41 | AY530604 |
| AAV12 | DQ813647 | AAV 3B | NC_001863 | Hu37 | AY530600 |
| AAV13 | EU285562 | Hu9 | AY530629 | Rh40 | AY530559 |
| | | Hu10 | AY530576 | Rh2 | AY243007 |
| Clade A | | Hu11 | AY530577 | Bb1 | AY243023 |
| AAV1 | NC_002077, | Hu53 | AY530615 | Bb2 | AY243022 |
| | AF063497 | | | | |
| AAV6 | NC_001862 | Hu55 | AY530617 | Rh10 | AY243015 |
| Hu.48 | AY530611 | Hu54 | AY530616 | Hu17 | AY530582 |
| Hu 43 | AY530606 | Hu7 | AY530628 | Hu6 | AY530621 |
| Hu 44 | AY530607 | Hu18 | AY530583 | Rh25 | AY530557 |
| Hu 46 | AY530609 | Hu15 | AY530580 | Pi2 | AY530554 |
| | | Hu16 | AY530581 | Pi1 | AY530553 |
| Clade B | | Hu25 | AY530591 | Pi3 | AY530555 |
| Hu19 | AY530584 | Hu60 | AY530622 | Rh57 | AY530569 |
| Hu20 | AY530586 | Ch5 | AY243021 | Rh50 | AY530563 |
| Hu23 | AY530589 | Hu3 | AY530595 | Rh49 | AY530562 |
| Hu22 | AY530588 | Hu1 | AY530575 | Hu39 | AY530601 |
| Hu24 | AY530590 | Hu4 | AY530602 | Rh58 | AY530570 |
| Hu21 | AY530587 | Hu2 | AY530585 | Rh61 | AY530572 |
| Hu27 | AY530592 | Hu61 | AY530623 | Rh52 | AY530565 |
| Hu28 | AY530593 | | | Rh53 | AY530566 |
| Hu29 | AY530594 | Clade D | | Rh51 | AY530564 |
| Hu63 | AY530624 | Rh62 | AY530573 | Rh64 | AY530574 |
| Hu64 | AY530625 | Rh48 | AY530561 | Rh43 | AY530560 |
| Hu13 | AY530578 | Rh54 | AY530567 | AAV8 | AF513852 |
| Hu56 | AY530618 | Rh55 | AY530568 | Rh8 | AY242997 |
| Hu57 | AY530619 | Cy2 | AY243020 | Rh1 | AY530556 |
| Hu49 | AY530612 | AAV7 | AF513851 | | |
| Hu58 | AY530620 | Rh35 | AY243000 | Clade F | |
| Hu34 | AY530598 | Rh37 | AY242998 | AAV9 (Hu14) | AY530579 |
| Hu35 | AY530599 | Rh36 | AY242999 | Hu31 | AY530596 |
| AAV2 | NC_001401 | Cy6 | AY243016 | Hu32 | AY530597 |
| Hu45 | AY530608 | Cy4 | AY243018 | | |
| Hu47 | AY530610 | | | | |
| Hu51 | AY530613 | | | | |
| Hu52 | AY530614 | | | | |
| Hu T41 | AY695378 | | | | |

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged. The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

The AAV viral vectors of the invention may include a recombinant AAV vector genome. A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s) but need not be contiguous thereto. The TRs can be the same or different from each other. The vector genome can also contain a single ITR at its 3′ or 5′ end. The terms “rAAV particle” and “rAAV virion” are used interchangeably here. A “rAAV particle” or “rAAV virion” comprises a rAAV vector genome packaged within an AAV capsid.

[The term “terminal repeat” or “TR” includes any viral terminal repeat or synthetic sequence that forms a hairpin structure and functions as an inverted terminal repeat (ITR) (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV terminal repeat” or “AAV TR” may be from any AAV, including but not limited to serotypes 1, 2, 3, 4, 5, 6, 7, 8, 9, 10, 11, 12, or 13 or any other AAV now known or later discovered (see, e.g., Table 3). An AAV terminal repeat need not have the native terminal repeat sequence (e.g., a native AAV TR sequence may be altered by insertion, deletion, truncation and/or missense mutations), as long as the terminal repeat mediates one or more of the desired functions, e.g., replication, virus packaging, integration, and/or provirus rescue, and the like.

Further, the viral capsid or genomic elements can contain other modifications, including insertions, deletions and/or substitutions.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wildtype or synthetic. A wildtype large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 3). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

In some embodiments, the transgene delivery vector encodes a protein or nucleic acid. In some embodiments, the protein is an enzyme, a regulatory protein, or a structural protein, e.g., one that can substitute for a missing or defective protein in a subject. In some embodiments, the nucleic acid is a functional nucleic acid, e.g., an antisense nucleic acid or an inhibitory RNA.

Any nucleic acid sequence(s) of interest may be delivered in the transgene delivery vectors of the present invention. Nucleic acids of interest include nucleic acids encoding polypeptides, including therapeutic (e.g., for medical or veterinary uses), immunogenic (e.g., for vaccines), or diagnostic polypeptides.

Therapeutic polypeptides include, but are not limited to, cystic fibrosis transmembrane regulator protein (CFTR), dystrophin (including mini- and micro-dystrophins (see, e.g., Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor −3 and −4, brain-derived neurotrophic factor, bone morphogenic proteins [including RANKL and VEGF], glial derived growth factor, transforming growth factor −α and −β, and the like), lysosomal acid α-glucosidase, α-galactosidase A, receptors (e.g., the tumor necrosis growth factorα soluble receptor), S100A1, parvalbumin, adenylyl cyclase type 6, a molecule that effects G-protein coupled receptor kinase type 2 knockdown such as a truncated constitutively active bARKct, anti-inflammatory factors such as IRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

Nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Alternatively, in particular embodiments of this invention, the nucleic acid may encode a functional nucleic acid, i.e., nucleic acid that functions without getting translated into a protein, e.g., an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan [e.g., α, β, γ], RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the nucleic acid may encode protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenic proteins (including BNP 2, 7, etc., RANKL and/or VEGF).

The transgene delivery vectors may also comprise a nucleic acid that shares homology with and recombines with a locus on a host chromosome. This approach can be utilized, for example, to correct a genetic defect in the host cell.

The present invention also provides transgene delivery vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, 5,863,541 to Samulski et al.). The antigen may be presented in the parvovirus capsid. Alternatively, the antigen may be expressed from a nucleic acid introduced into a recombinant vector genome. Any immunogen of interest as described herein and/or as is known in the art can be provided by the nucleic acid delivery vectors.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) (ancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

It will be understood by those skilled in the art that the nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements or cell stress-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the initiation codon (e.g., ATG) and adjacent sequences, can be of a variety of origins, both natural and synthetic.

The cell(s) into which the transgene delivery vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above. Furthermore, the cells may be dividing or non-dividing.

Embodiments of the invention may be performed in vitro or in vivo. One aspect of the present invention is a method of expressing a transgene in a cell in vitro, e.g., for research purposes or as part of an ex vivo method. The transgene delivery vector may be introduced into the cells at the appropriate amount, e.g., multiplicity of infection for a viral vector, according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

In particular embodiments, the cells have been removed from a subject, the transgene delivery vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the transgene delivery vectors can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the transgene delivery vector are administered to the subject in a treatment effective or prevention effective amount in combination with a pharmaceutical carrier.

The transgene delivery vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The transgene delivery vectors can also be used to produce a polypeptide of interest or functional RNA in a subject (e.g., using the subject as a bioreactor to produce the polypeptide or to observe the effects of the functional nucleic acid on the subject, for example, in connection with screening methods). The transgene delivery vectors may also be employed to provide a functional nucleic acid to a cell in vitro or in vivo. Expression of the functional nucleic acid in the cell, for example, can diminish expression of a particular target protein by the cell. Accordingly, functional nucleic acid can be administered to decrease expression of a particular protein in a subject in need thereof.

Transgene delivery vectors also find use in diagnostic and screening methods, whereby a nucleic acid of interest is transiently or stably expressed in a transgenic animal model.

The transgene delivery vectors can also be used for various non-therapeutic purposes, including but not limited to use in protocols to assess gene targeting, clearance, transcription, translation, etc., as would be apparent to one skilled in the art. The transgene delivery vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

### Pharmaceutical Formulations, Subjects, and Modes of Administration

Provided according to embodiments of the invention are compositions that include a transgene delivery vector. Also provided herein are pharmaceutical compositions comprising a transgene delivery vector in a pharmaceutically acceptable carrier and, optionally, other medicinal agents, pharmaceutical agents, stabilizing agents, buffers, carriers, adjuvants, diluents, etc. In addition to the transgene delivery vector, the fusion protein and/or CEM of the invention may be present in the same pharmaceutical composition as the transgene delivery vector or in separate pharmaceutical compositions. For injection, the carrier will typically be a liquid. For other methods of administration, the carrier may be either solid or liquid. For inhalation administration, the carrier will be respirable, and optionally can be in solid or liquid particulate form. By “pharmaceutically acceptable” it is meant a material that is not toxic or otherwise undesirable, i.e., the material may be administered to a subject without causing any undesirable biological effects.

A further aspect of the invention is a method of administering the transgene delivery vector, fusion protein, and/or CEM to subjects. Administration of the transgene delivery vectors, fusion proteins, and/or CEMs to a human subject or an animal in need thereof can be by any means known in the art. Optionally, the transgene delivery vector, fusion protein, and/or CEM is delivered in a treatment effective or prevention effective dose in a pharmaceutically acceptable carrier.

Injectables can be prepared in conventional forms, either as liquid solutions or suspensions, solid forms suitable for solution or suspension in liquid prior to injection, or as emulsions. Alternatively, one may administer the transgene delivery vectors, fusion proteins, and/or CEMs in a local rather than systemic manner, for example, in a depot or sustained-release formulation. Further, the transgene delivery vectors, fusion proteins, and/or CEMs can be delivered adhered to a surgically implantable matrix (e.g., as described in U.S. Patent Publication No. 2004-0013645). The transgene delivery vectors, fusion proteins, and/or CEMs disclosed herein can be administered to the lungs of a subject by any suitable means, optionally by administering an aerosol suspension of respirable particles comprised of the transgene delivery vectors, fusion proteins, and/or CEMs, which the subject inhales. The respirable particles can be liquid or solid. Aerosols of liquid particles comprising the transgene delivery vectors, fusion proteins, and/or CEMs may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the transgene delivery vectors, fusion proteins, and/or CEMs may likewise be produced with any solid particulate medicament aerosol generator, by techniques known in the pharmaceutical art.

In certain embodiments, the transgene delivery vectors, fusion proteins, and/or CEMs are administered to a subject in need thereof as early as possible in the life of the subject, e.g., as soon as the subject is diagnosed with a disease or disorder. In some embodiments, the methods are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder. In some embodiments, the methods are carried out on a subject before the subject develops symptoms associated with a disease or disorder, e.g., a subject that is suspected or diagnosed as having a disease or disorder but has not started to exhibit symptoms.

The transgene delivery vectors, fusion proteins, and/or CEMs may be administered to a subject by any route of administration found to be effective to regulate transgene expression in the host cell. The most suitable route will depend on the subject being treated and the disorder or condition being treated. In some embodiments, the transgene delivery vectors, fusion proteins, and/or CEMs are administered to the subject by a route selected from oral, rectal, transmucosal, intranasal, inhalation (e.g., via an aerosol), buccal (e.g., sublingual), vaginal, intrathecal, intraocular, intravitreal, intracochlear, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye (e.g., by intrastromal, topical, intracameral, intravitreal, subconjunctival, suprachoroidal, sub-Tenon, retrobulbar, or subretinal administration), skeletal muscle, cardiac muscle, diaphragm muscle or brain (e.g., by intrathecal, intracerebral, intraventricular, intranasal, intra-aural, intra-ocular, or peri-ocular delivery administration)).

In particular embodiments, more than one administration (e.g., two, three, four or more administrations) may be employed to achieve the desired level of gene expression over a period of various intervals, e.g., daily, weekly, monthly, yearly, etc.

The transgene delivery vectors, fusion proteins, and/or CEMs can be administered to tissues of the CNS (e.g., brain, eye) and may advantageously result in broader distribution of the transgene delivery vectors, fusion proteins, and/or CEMs than would be observed in the absence of the present invention.

Administration can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoideohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The transgene delivery vectors, fusion proteins, and/or CEMs can be delivered to skeletal muscle by intravenous administration, intra-arterial administration, intraperitoneal administration, limb perfusion, (optionally, isolated limb perfusion of a leg and/or arm; see, e.g., Arruda et al., (2005) Blood 105: 3458-3464), and/or direct intramuscular injection. In particular embodiments, the transgene delivery vectors, fusion proteins, and/or CEMs are administered to a limb (arm and/or leg) of a subject (e.g., a subject with muscular dystrophy such as DMD) by limb perfusion, optionally isolated limb perfusion (e.g., by intravenous or intra-articular administration. In embodiments of the invention, the transgene delivery vectors, fusion proteins, and/or CEMs can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the agent to cross the endothelial cell barrier. In particular embodiments, the transgene delivery vectors, fusion proteins, and/or CEMs can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The transgene delivery vectors, fusion proteins, and/or CEMs can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Administration to smooth muscle can be by any suitable method including intravenous administration, intra-arterial administration, and/or intra-peritoneal administration. In one embodiment, administration can be to endothelial cells present in, near, and/or on smooth muscle.

Delivery to a target tissue can also be achieved by delivering a depot comprising the transgene delivery vectors, fusion proteins, and/or CEMs. In representative embodiments, a depot comprising the transgene delivery vectors, fusion proteins, and/or CEMs is implanted into skeletal, smooth, cardiac and/or diaphragm muscle tissue or the tissue can be contacted with a film or other matrix comprising the heterologous agent. Such implantable matrices or substrates are described in U.S. Pat. No. 7,201,898.

Administration can also be to a tumor (e.g., in or near a tumor or a lymph node). The most suitable route in any given case will depend on the nature and severity of the condition being treated and/or prevented and on the nature of the particular vector that is being used.

The transgene delivery vectors, fusion proteins, and/or CEMs may be delivered or targeted to any tissue or organ in the subject. The target tissue or organ may be in vivo or ex vivo (e.g., corneas or other tissues for transplantation). In some embodiments, the transgene delivery vectors, fusion proteins, and/or CEMs are administered to, e.g., a skeletal muscle, a smooth muscle, the heart, the diaphragm, the airway epithelium, the liver, the kidney, the spleen, the pancreas, the skin, the lung, the ear, and the eye. In some embodiments, the transgene delivery vectors, fusion proteins, and/or CEMs are administered to a diseased tissue or organ, e.g., a tumor.

In general, the transgene delivery vectors of the present invention can be employed to deliver a nucleic acid encoding a polypeptide or functional nucleic acid to treat and/or prevent any disease state for which it is beneficial to deliver a therapeutic polypeptide or functional nucleic acid. Illustrative disease states include, but are not limited to: cystic fibrosis (cystic fibrosis transmembrane regulator protein) and other diseases of the lung, hemophilia A (Factor VIII), hemophilia B (Factor IX), thalassemia (ß-globin), anemia (erythropoietin) and other blood disorders, Alzheimer's disease (GDF; neprilysin), multiple sclerosis (ß-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects, congenital emphysema (α1-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). In one example, HLA-G isoforms may be administered. As another example, bone morphogenic proteins (including BNP 2, 7, etc., RANKL and/or VEGF) can be administered with a bone allograft, for example, following a break or surgical removal in a cancer patient.

In particular embodiments, a transgene delivery vectors are administered to skeletal muscle, diaphragm muscle and/or cardiac muscle (e.g., to treat and/or prevent muscular dystrophy or heart disease [for example, PAD or congestive heart failure]).

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, transgene delivery vectors permit the treatment and/or prevention of genetic diseases.

As a further aspect, the transgene delivery vectors of the present invention may be used to produce an immune response in a subject. According to this embodiment, transgene delivery vectors comprising a nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the transgene delivery vectors may be administered to a cell ex vivo and the altered cell is administered to the subject. The transgene delivery vectors comprising the nucleic acid is introduced into the cell, and the cell is administered to the subject, where the nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof. In particular embodiments, the nucleic acid delivery vector or cell comprising the nucleic acid can be administered in an immunogenically effective amount, as described below.

The transgene delivery vectors can also be administered for cancer immunotherapy by administration of transgene delivery vectors expressing one or more cancer cell antigens (or an immunologically similar molecule) or any other immunogen that produces an immune response against a cancer cell. To illustrate, an immune response can be produced against a cancer cell antigen in a subject by administering transgene delivery vectors comprising a nucleic acid encoding the cancer cell antigen, for example to treat a patient with cancer and/or to prevent cancer from developing in the subject. The transgene delivery vectors may be administered to a subject in vivo or by using ex vivo methods, as described herein. Alternatively, the cancer antigen can be expressed as part of the transgene delivery vectors.

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with transgene delivery vectors. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the transgene delivery vectors.

Cytokines may be administered by any method known in the art. Exogenous cytokines may be administered to the subject, or alternatively, a nucleic acid encoding a cytokine may be delivered to the subject using a suitable vector, and the cytokine produced in vivo.

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a polynucleotide including those described herein. As a further option, the subject can be a laboratory animal and/or an animal model of disease. Preferably, the subject is a human.

In some embodiments, the transgene delivery vectors are introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

The transgene delivery vectors can further be administered to elicit an immunogenic response (e.g., as a vaccine). Typically, immunogenic compositions of the present invention comprise an immunogenically effective amount of transgene delivery vectors in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the transgene delivery vectors to be administered to a subject depend upon the mode of administration, the disease or condition to be treated and/or prevented, the individual subject's condition, the particular transgene delivery vector, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 101⁸ transducing units, optionally about 10⁸-10¹⁵ transducing units.

In a representative embodiment, the invention provides a method of treating and/or preventing muscular dystrophy in a subject in need thereof, the method comprising: administering a treatment or prevention effective amount of transgene delivery vectors to a mammalian subject, wherein the transgene delivery vector comprises a nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the transgene delivery vectors can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional nuclei acid (e.g., functional RNA, e.g., RNAi, microRNA, antisense RNA) that normally circulates in the blood or for systemic delivery to other tissues to treat and/or prevent a disorder (e.g., a metabolic disorder, such as diabetes (e.g., insulin), hemophilia (e.g., Factor IX or Factor VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid α glucosidase]). Other suitable proteins for treating and/or preventing metabolic disorders are described above. The use of muscle as a platform to express a nucleic acid of interest is described in U.S. Patent Publication No. 2002/0192189.

Thus, as one aspect, the invention further encompasses a method of treating and/or preventing a metabolic disorder in a subject in need thereof, the method comprising: administering a treatment or prevention effective amount of transgene delivery vectors to a subject (e.g., to skeletal muscle of a subject), wherein the transgene delivery vector comprises a nucleic acid encoding a polypeptide, wherein the metabolic disorder is a result of a deficiency and/or defect in the polypeptide. Illustrative metabolic disorders and nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering heterologous agent and the cell membrane fusion protein or a functional fragment or derivative thereof to skeletal muscle are described in more detail herein.

The invention can also be practiced to produce antisense RNA, RNAi or other functional RNA (e.g., a ribozyme) for systemic or local delivery.

The invention also provides a method of treating and/or preventing congenital heart failure or PAD in a subject in need thereof, the method comprising administering a treatment or prevention effective amount of transgene delivery vectors to a mammalian subject, wherein the heterologous agent comprises a nucleic acid encoding, for example, a sarcoplasmic endoreticulum Ca2⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206 and/or mir-208.

In particular embodiments, the transgene delivery vectors may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The transgene delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of transgene delivery vectors encoding one or more neurotrophic factors.

Age-related macular degeneration involves both angiogenesis and retinal degeneration. This disorder can be treated by administering transgene delivery vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the transgene delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using transgene delivery vectors of the invention to treat a pituitary tumor. According to this embodiment, the transgene delivery vectors encoding somatostatin (or an active fragment thereof) are administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art.

In particular embodiments, the transgene delivery vectors can comprise a secretory signal as described in U.S. Pat. No. 7,071,172.

In representative embodiments of the invention, the transgene delivery vectors are administered to the CNS (e.g., to the brain or to the eye). The transgene delivery vectors may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The transgene delivery vectors may also be administered to different regions of the eye such as the retina, cornea and/or optic nerve.

The transgene delivery vectors may be delivered into the cerebrospinal fluid (e.g., by lumbar puncture) for more disperse administration of the transgene delivery vectors. The transgene delivery vectors may further be administered intravascularly to the CNS in situations in which the blood-brain barrier has been perturbed (e.g., brain tumor or cerebral infarct).

The transgene delivery vectors can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the transgene delivery vectors are administered in a liquid formulation by direct injection (e.g., stereotactic injection) to the desired region or compartment in the CNS. In other embodiments, the transgene delivery vectors may be provided by topical application to the desired region or by intra-nasal administration of an aerosol formulation. Administration to the eye, may be by topical application of liquid droplets. As a further alternative, the transgene delivery vectors may be administered as a solid, slow-release formulation (see, e.g., U.S. Pat. No. 7,201,898).

In yet additional embodiments, the transgene delivery vectors can used for retrograde transport to treat and/or prevent diseases and disorders involving motor neurons (e.g., amyotrophic lateral sclerosis (ALS); spinal muscular atrophy (SMA), etc.). For example, the transgene delivery vectors can be delivered to muscle tissue or parts of the eye (e.g., anterior ocular segment or eyelid-associated glands) from which it can migrate into neurons.

Having described the present invention, the same will be explained in greater detail in the following examples, which are included herein for illustration purposes only, and which are not intended to be limiting to the invention.

### EXAMPLES

### Example 1

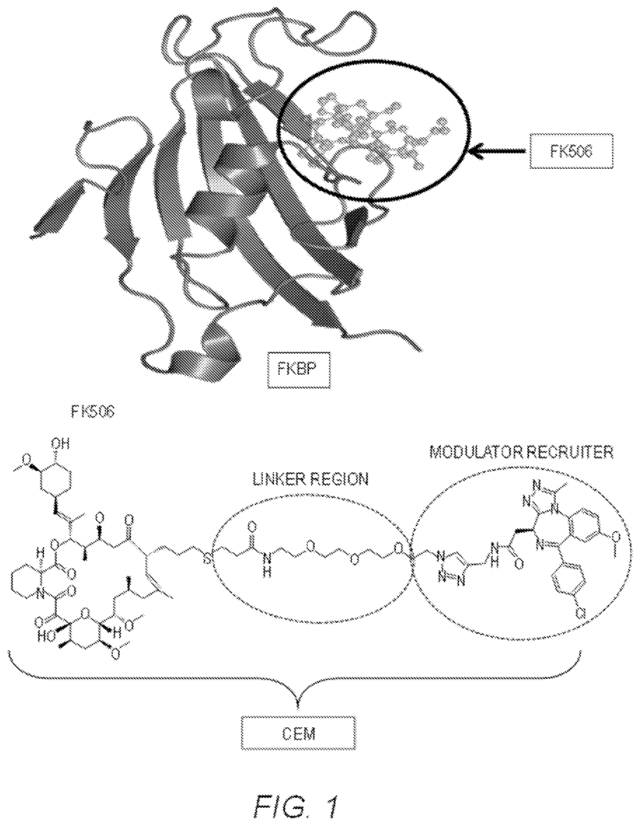

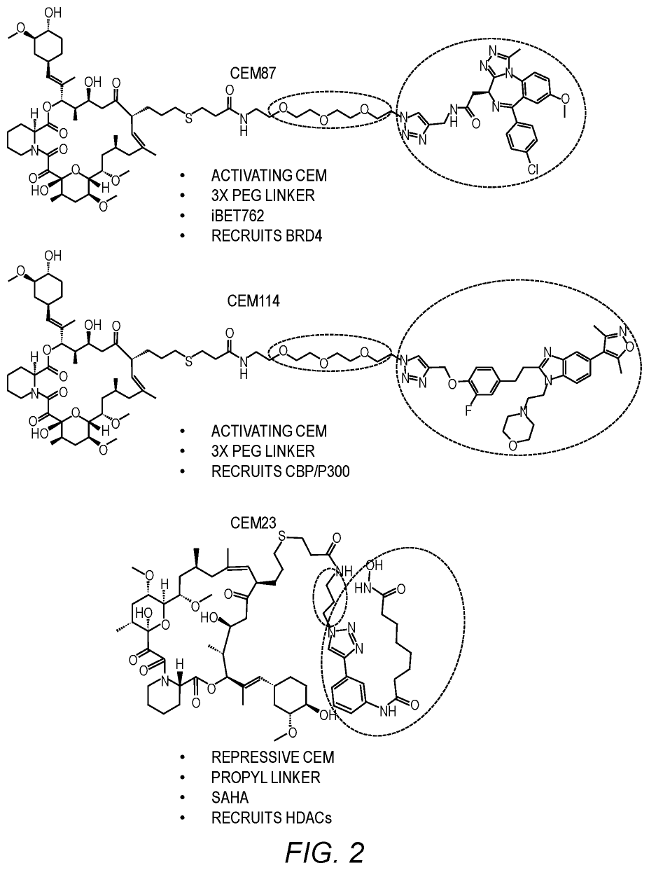

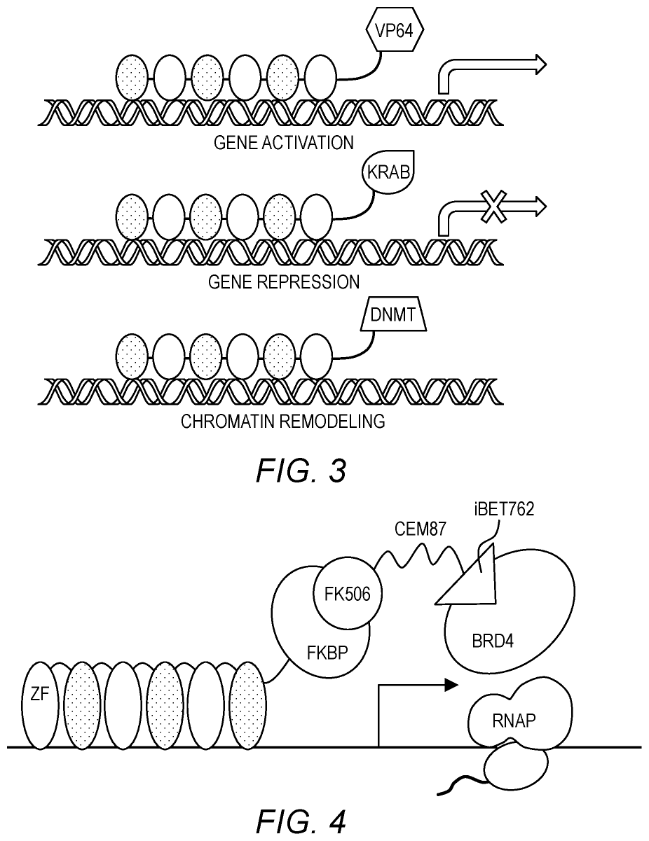

The inventors have pioneered a powerful new technology that leverages a combination of a ZF DNA binding module coupled with added chemical control systems that harness endogenous host enzymes to control gene transcription in a dose-dependent, as well as reversible, fashion. By using a DNA binding domain to target a gene-of-interest, CEMs localize to the gene locus and recruit endogenous chromatin modifying enzymes to modulate transcription (Butler et al., ACS Synth. Biol. 7(1):38 (2018); Chiarella et al., J. Vis. Exp. 2018(139); Chiarella et al., Nat. Biotechnol. 38(1):50 (2020)). The inventors have demonstrated the CEM platform is a powerful tool to control chromosomal gene regulation and investigated this system on transduced AAV episomes towards controlled therapies (Butler et al., ACS Synth. Biol. 7(1):38 (2018); Chiarella et al., J. Vis. Exp. 2018(139); Chiarella et al., Nat. Biotechnol. 38(1):50 (2020)). The general structure of CEMs is depicted in FIG. 1. Representative structures of activating and repressive CEMs are shown in FIG. 2. Examples of the use of zinc finger proteins to modulate chromatin and expression are shown in FIGS. 3-4. Zinc finger proteins (ZFs) are endogenous small (˜30 amino acid) proteins that can bind 3 base pairs of DNA. Six ZFs linked together achieve DNA sequence specificity. Synthetic ZF arrays have been used for targeted gene editing and targeted gene regulation.

Mechanistically, the chromatinization of AAV vector episomes has been understudied, however, preliminary data demonstrate that they are repressed, limiting therapeutic efficacy and necessitating high dose clinical AAV gene therapy. The ability to precisely and transiently modulate transgene production in vivo would increase the safety of AAV gene therapy in general, as particular transgene derived products in overabundance may elicit unwanted toxicity. In addition, the ability to control transgene production in vivo offers the potential to evade the immune response to transgenic products implicated in the failures of clinical approaches.

CEM Activation of AAV Vectors in vitro: In preliminary studies, activation of AAV episomes by 2 different CEMs was observed. In these studies, a well described Zinc Finger (ZF) binding domain, ZFHD1 (Hathaway et al., Cell 149(7):1447 (2012)), and its cognate binding sequence engineered on the AAV genomes was used to provide CEM specificity.

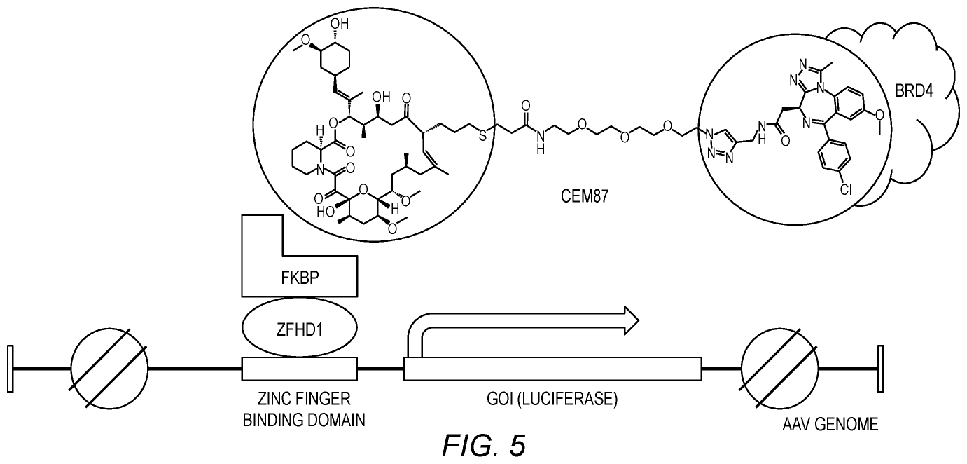

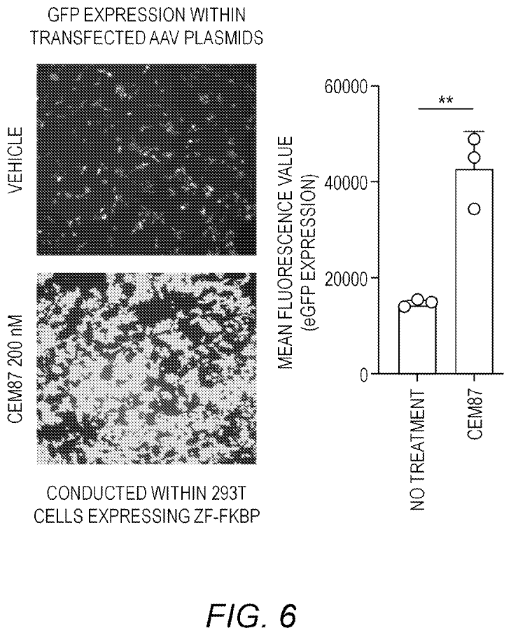

The first experiment examined whether CEM87 can control gene expression of transfected plasmids. HEK293T.17 cells that stably express ZF-FKBP (a zinc finger array (ZF) fused to FK506 Binding Protein (FKBP)) were transfected with a plasmid that contains a zinc finger DNA binding domain (i.e., zinc finger recognition sequence) specific to the array in ZF-FKBP upstream of a JET promoter and an eGPF reporter gene downstream of the promoter (FIG. 5). After 16 h the cell media was replaced with media containing either 200 nM CEM87 or a DMSO control. 48 hours post CEM87 addition, cells were imaged and flow cytometry run to determine the eGFP expression. The results are shown in FIG. 6, left panel.

It was then examined whether CEM87 can control recombinant AAV transgene expression in transduced cells. HEK293T.17 cells that stably expresses ZF-FKBP were transduced with an AAV2 vector that contains a zinc finger DNA binding domain recognition sequence specific to the array in ZF-FKBP upstream of a JET promoter and an eGPF reporter gene downstream of the promoter. After 24 h the cell media was replaced with media containing either 200 nM CEM87 or a DMSO control. 48 hours post CEM87 addition, cells were imaged and flow cytometry run to determine the percent eGFP expression. The results are shown in FIG. 6, right panel.

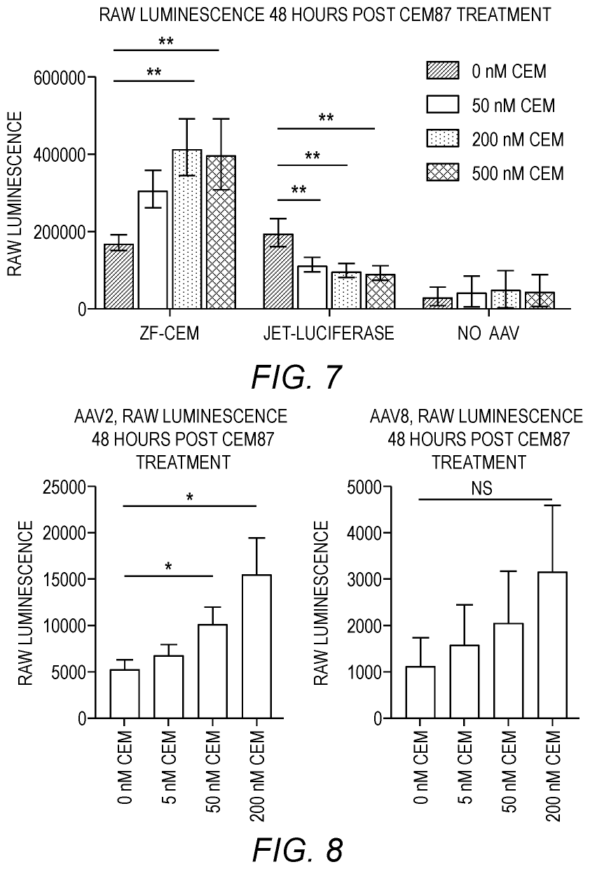

An AAV vector expressing both the ZF-FKBP fusion protein and luciferase was tested. HEK293T.17 cells were transduced with an AAV2 vector that contains a zinc finger DNA binding domain recognition sequence specific to the array in ZF-FKBP upstream of a JET promoter and the ZF-FKBP gene directly followed by the luciferase reporter gene downstream of the promoter (ZF-CEM in FIG. 7). Controls included an AAV2 vector missing the ZF-FKBP gene (Jet-Luciferase) and no transduction (No AAV). After 16 h the cell media was replaced with media containing either CEM87 (5 nM, 50 nM, or 200 nM) or a DMSO control. 48 hours post CEM87 addition, a luciferase assay (Promega) was run to determine luciferase expression. The results are shown in FIG. 7.

It was next tested whether CEM87 can control transduced recombinant AAV transgene expression in two different AAV serotypes (AAV2 and AAV8). HEK293T.17 cells were transduced with an AAV vector that contains a zinc finger DNA binding domain recognition sequence specific to the array in ZF-FKBP upstream of a JET promoter and the ZF-FKBP gene directly followed by the luciferase reporter gene downstream of the promoter. After 24 h the cell media was replaced with media containing either CEM87 (5 nM, 50 nM, or 200 nM) or a DMSO control. 48 hours post CEM87 addition, a luciferase assay (Promega) was run to determine luciferase expression. The results are shown in FIG. 8.

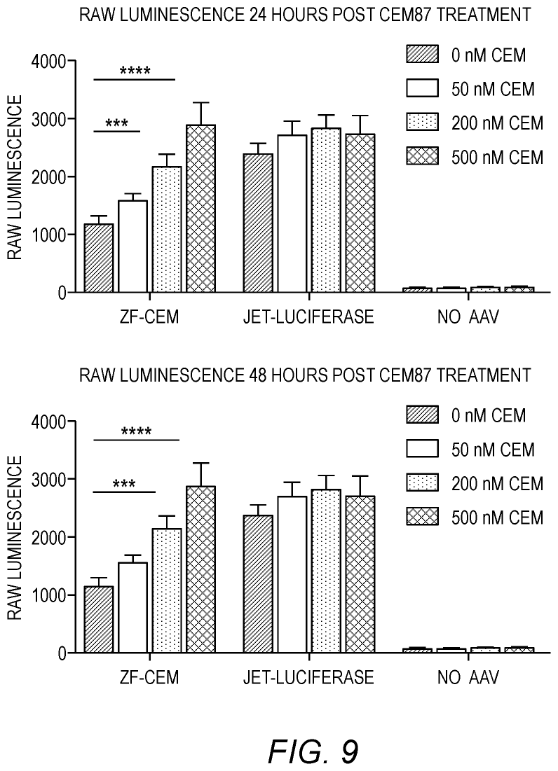

It was then examined whether CEM87 can control transduced recombinant AAV (AAV2) transgene expression at 24 and 48 hours post CEM87 addition. HEK293T.17 cells were transduced with an AAV2 vector that contains a zinc finger DNA binding domain recognition sequence specific to the array in ZF-FKBP upstream of a JET promoter and the ZF-FKBP gene directly followed by the luciferase reporter gene downstream of the promoter (ZF-CEM in FIG. 9). Controls included a plasmid that only contains a JET promoter and a downstream luciferase reporter gene (Jet-Luciferase) and no transduction (No AAV). After 24 h the cell media was replaced with media containing either CEM87 (50 nM, 200 nM, or 500 nM) or a DMSO control. 24 and 48 hours post CEM87 addition, a luciferase assay (Promega) was run to determine luciferase expression. The results are shown in FIG. 9.

Some of these experiments relied on a 293 cell line harboring a stable constitutive ZF-FKBP cassette (293-ZF-FKBP) and transduction by AAV2-JET-GFP vectors harboring the ZF binding domain recognition sequence (BD) upstream of the constitutive JET promoter (FIG. 5). Two chemicals were investigated in these initial studies, CEM87 and CEM114 which bind ZF-FKBP and act to recruit BRD4 or CBP/p300, respectively. To date, no associations of BRD4 and transduced AAV genomes have been reported, however, CBP/p300 was shown to precipitate with AAV vector genomes recovered from heart tissue with speculation of a role in enhanced transgene transcription (Dean et al., Hum. Gene Ther. 20(9): 1005 (2009)). Following the transduction of AAV2-BD-Jet-GFP vectors to 293-ZF-FKBP cells, increasing concentrations of CEMs (87 or 114) were added to the culture medium. Two days later, GFP fluorescence was analyzed by flow cytometry. The data demonstrate dose-dependent activation of AAV2-BD-JET-GFP genomes >10-fold in response to CEM87 (FIG. 10). Control experiments using AAV vectors without the engrafted ZF BD or AAV2-BD-JET-GFP in WT 293 cells demonstrated no drug response highlighting the specificity of transgene activation (FIG. 10). From a mechanistic standpoint, the CEM87 data suggest that AAV episomes are responsive to transcriptional enhancement by BRD4 or CBP/p300 resulting in the hypothesis that transduced AAV episomes are not completely silenced yet conversely, not maintained in a fully “ON” chromatin configuration either (as they can be activated by CEMs).

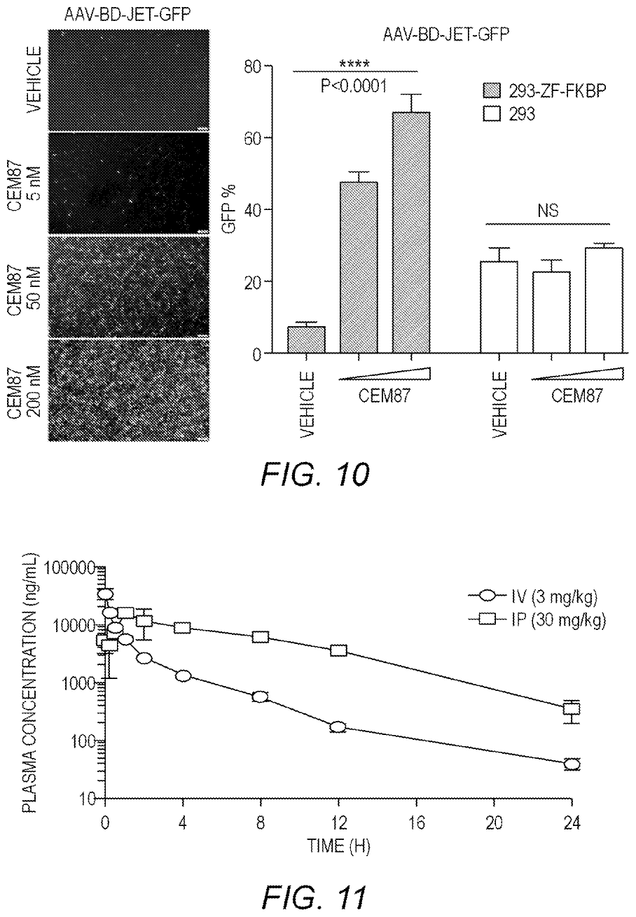

Pharmacokinetic Data of CEM87 in Mice: Initial and encouraging pharmacokinetics (PK) analysis of CEM87 were conducted. Mice were administered a single 3 mg/kg dose of CEM87 via intravenous (IV) injection or a single dose of 30 mg/kg of CEM87 through the intraperitoneal (IP) route (n=9 per group, 3 mice per time point). Mice treated with 3 mg/kg IV had a maximal concentration (Cₘₐₓ) of 34,069 ng/mL, along with exposure (AUC₀₋₂₄) of 27,506 h*ng*mL−1. CEM87 in this route had a half-life (t_{1/2}) of roughly 4 hrs (FIG. 11). 30 mg/kg of CEM87 administration by IP resulted in ˜50% bioavailability (FIG. 11). Mice administered with 30 mg/kg by IP had a Cₘₐₓ of 15,489 ng/mL, an AUC₀₋₂₄ of 114,422 h*ng*mL−1, and finally a t_{1/2} of 4 hrs, indicating linear PK (FIG. 11).

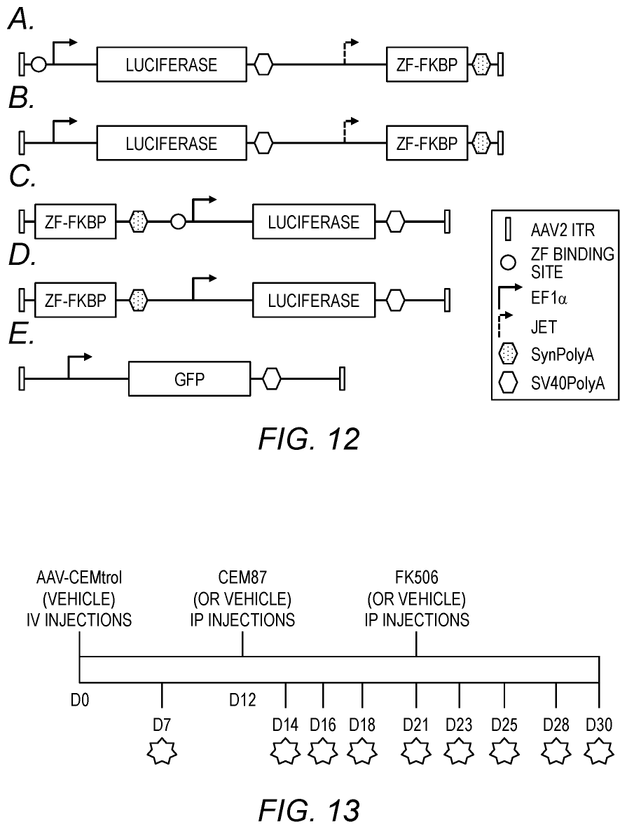

AAV vector platform contexts for chemical control of transgene expression: Preliminary data demonstrates the capacity for ZF-FKBP to regulate transduced episomal AAV vector gene expression specifically when the ZF BD is engrafted upstream of the promoter in the vector cassette (FIG. 10). In those experiments, the ZF-FKBP protein was supplied in trans from a lentiviral chromosomal integrant. For envisioned gene therapy applications, it is necessary to encode this small (800 nt) host-like fusion protein on the AAV vector generating a platform vector context for controlled AAV transgene expression via chemical induced epigenetic control (termed AAV-CEMtrol). Two genetic cassettes that fulfill this requirement are depicted, along with the proper control vectors (FIG. 12). In the first construct, ZF-FKBP transcription relies on a small (230 nt) synthetic JET promoter that confers ubiquitous transcription at a relatively moderate to low level (construct A) (Llanga et al., Mol. Ther. 25(9):2150 (2017)). A second strong ubiquitous promoter (EF1α) transcribes the gene of interest, in this case luciferase. This two promoter AAV transgenic configuration is considered the conservative approach for the necessary components of AAV-CEMtrol. The second, novel, AAV-CEMtrol platform context (construct C) relies on the inventors' recent characterization of the AAV inverted terminal repeat (ITR), which confers low level transcriptional activity that is hypothesized sufficient for ZF-FKBP expression (Earley et al., Hum. Gene Ther. 31(3-4):151 (2020)). The advantage of this construct is that it allows greater AAV packaging capacity for the desired promoter and transgene, as an additional promoter for ZF-FKBP expression is not required. This context will also test the hypothesis that low level ZF-FKBP transcription will supply excess regulator protein to control the episomal AAV vector genomes upon CEM induction. For both contexts, versions without the ZF-FKBP binding site upstream of the luciferase reporter (EF1α) will serve as negative controls (constructs B, D). Additionally, an AAV-GFP transgenic sequence will serve as the luciferase negative control (construct E).

Chemical activation and subsequent repression of AAV2-CEMtrol in vitro: In preliminary evidence, CEM87 demonstrated dose-dependent activation of AAV-CEMtrol-GFP expression up to 10-fold, specifically in the transgenic cassette containing the ZF-FKBP binding site (FIG. 10). This result suggests that BRD4 recruitment to transduced episomes by CEM87 is sufficient for AAV transgene activation, however, the ability to “Turn OFF” AAV vector expression via ZF-FKBP binding competition between CEM87 and the FDA approved chemical FK506 was not investigated. Therefore, using the constructs depicted in FIG. 12, CEM87 activation of AAV-CEMtrol followed by FK506, which binds to FKBP in a competitive manner to release CEM87 (Stanton et al., Science 359(6380) (2018)), and is hypothesized to repress reporter transcription, will be investigated in human cell culture. For all studies, AAV2 vectors will be administered to 293 cells at a dose of 500 viral genomes (vg) per cell 24 h prior to CEM87 addition at a concentration of 200 nM (N=8 per group). Two days later, fresh medium will be added along with the addition of 500 nM FK506 which may reverse the interaction of CEM87 with ZF-FKBP. After 48 h of culture, cells will be harvested and analyzed for luciferase activity which is then normalized to total recovered protein (Miyadera et al., Mol. Ther. 28(6):1455 (2020)). Constructs with no ZF-FKBP binding site will serve as CEM87 activation negative controls, an AAV2-GFP vector will serve as the negative control for luciferase activity, and the vehicle will act as a control for CEM87 and FK506. The optimal AAV-CEMtrol platform cassette identified herein is defined as the cassette that demonstrates the greatest level of activation in the presence of the drug compared to the no drug, non-induced state.

AAV episome immunoprecipitation #peak CEM87 concentration: To evaluate if effects are due to on-target effects, modifications to the episomal epigenome will be examined. Chromatin immunoprecipitation (ChIP) followed with qPCR will be conducted to examine levels of BRD4 (target of CEM87) and RNA PolII. These experiments will be conducted at both 24- and 48-hours following CEM87 addition, again using vehicle as one control and no ZF-FKBP as the second control. qPCR primers will be designed to test regions of the promoter and the luciferase gene itself in all AAV vectors. A non-targeted region of AAV as well as host genomic control primers will be used to calibrate qPCR properly for relative enrichment. This will determine the extent to which the desired host epigenetic activator (BRD4) is recruited as well as the level of transcriptional machinery (represented by RNA PolII) present due to CEM87 addition compared to controls.

AAV transgene regulation in multiple tissues following CEM87 administration: Preliminary data have demonstrated that the CEM87/ZF-FKBP recruitment of BRD4 to transduced AAV episomes rapidly enhances transgene expression >10-fold (FIG. 10). An optimized AAV-CEMtrol platform cassette will be selected that expresses both the ZF-FKBP fusion protein and a luciferase ORF under the control of a chosen promoter (EF1α) that is preceded by the ZF binding sequence (FIG. 12). This AAV-CEMtrol format allows ZF-FKBP transgene modulation in response to CEM87 binding (FIG. 10). Rigorous, well controlled, and logical in vivo studies will be conducted testing the capacity for AAV-CEMtrol regulation in multiple tissues to determine the potential for such a system towards safer controlled gene therapy. Importantly, PK studies demonstrate the bioavailability of CEM87 in mice and along with the in vitro data support the hypothesis that the AAV-CEMtrol vector developed herein will regulate AAV transgene expression in vivo in response to CEM87 administration (FIGS. 10-11).

AAV9-CEMtrol production and vector validations: These investigations will rely on the AAV9 capsid which mediates total body transduction in murine models and the optimized AAV-CEMtrol genome configuration characterized and defined above (FIG. 12) (Zincarelli et al., Mol. Ther. 16(6):1073 (2008)). These experiments will utilize the strong and ubiquitous EF1α promoter and a firefly luciferase reporter which allows live imaging of AAV vector transduction in multiple tissues in a kinetic manner (FIG. 12). AAV9-CEMtrol vectors will be obtained from the UNC Vector core which produces “clinical-like” grade vectors that are quality controlled for genome titer by qPCR and empty:full capsid ratios by silver staining (Grieger et al., Nat. Protoc. 1(3): 1412 (2006)). Once obtained, the AAV vectors will be aliquoted in low retention tubes for storage at −80° ° C. A vector sample will be analyzed independently for vector titer by qPCR using a minimal of 2 transgenic primer sets with probe detection and the results will be further validated by Southern dot blot analysis (Grieger et al., Nat. Protoc. 1(3): 1412 (2006)). Additionally, alkaline gel electrophoresis followed by Southern analysis will be used to determine the AAV genome integrity prior to application to animal models.

AAV9-CEMtrol and CEM87 administration: To ensure adequate sample size and power, the parameter of luminescence was used as evaluated in previous murine studies using AAV-luciferase vectors analyzed by the same IVIS live imaging machine used herein. Based on these studies, expected differences to detect changes with and without CEM87 induction is a relative luminescence (RLUs) of 2,000 with a SD of +/−800 RLUs. To detect a difference in luminescence between the vehicle or CEM87 treated animals with a minimum statistical power of 0.8, the group size needs to be a minimum of 6 animals (calculated using JMP Pro 14, SAS Institute, Inc). Therefore, group size herein will employ 12 replicates allowing for an even sex distribution and additionally, the potential to determine significant differences in the gender response. Prior to all experiments, serum will be collected from all animals to confirm the experimental subjects are negative for AAV9 capsid neutralizing antibodies (NABs) [23]. AAV9-CEMtrol (or vehicle) will be administered to 6 week old C57B6 mice of both gender at a dose of 1×10¹¹ viral genomes (vg) in a 100 μl final volume via IV injection. The overall experimental design is depicted in Table 4 and in FIG. 13. Washout experiments with FK506, which is also known as Tacrolimus an FDA approved drug, will be used at 5 mg/kg in line with the range of activity in published work.

TABLE 4

| | 1^{st} IP Injection | 2^{nd} IP | Replicates |
|---|---|---|---|
| Construct | (30 mg/kg) | Injection | (equal gender) |
| Vehicle | Vehicle | Vehicle | 12 |
| AAV9-luc (1e¹¹ vg) | Vehicle | Vehicle | 12 |
| AAV9-luc (1e¹¹ vg) | CEM87 | Vehicle | 12 |
| AAV9-luc (1e¹¹ vg) | Vehicle | FK506 | 12 |
| AAV9-luc (1e¹¹ vg) | CEM87 | FK506 | 12 |

Kinetic analysis of AAV transgene expression using live imaging: Live imaging of vector derived luminescence is measured using the IVIS lumina small animal imaging system. Briefly, D-luciferin solution is injected IP 5 min prior to detection of light emission. Imaging, under transient isoflurane anesthetization, of luciferase activity will be performed starting 7 days following vector injection and at the indicated intervals over a 1 month period (experimental conclusion). The CEM87 or vehicle injection at a dose of 30 mg/kg (FIGS. 11, 13) will be performed via IP injection on Day 12 after the vector injection (Table 4, FIG. 13). The FDA approved chemical FK506 which competes for FKBP binding, that harnesses endogenous Histone Deacetylase (HDAC) activity, will be investigated to actively repress the reporter locus will also be investigated. Following the CEM87 injection live imaging will occur as depicted (FIG. 13).

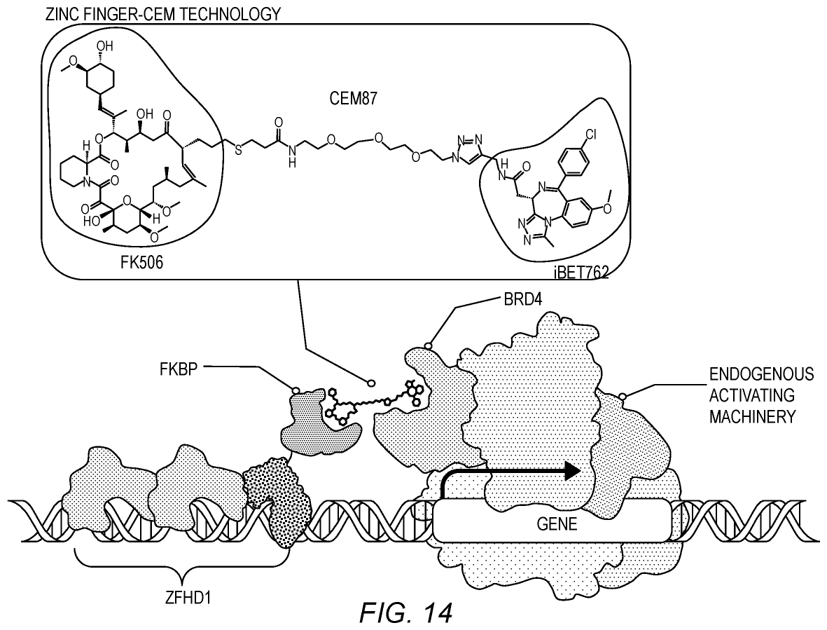

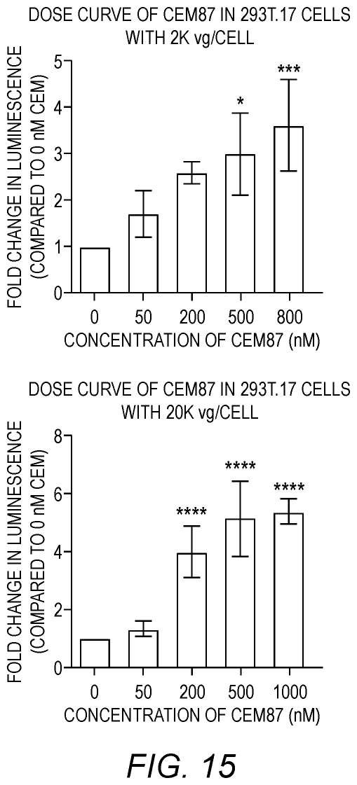

A summary of the ZF-CEM technology strategy for the following experiments is shown in FIG. 14. ZF-CEM Technology exhibited dose dependent chemical control with CEM87 at both low and high viral genomes per cell (FIG. 15). At a higher viral genomes per cell dose of virus, higher fold change in transgene (firefly luciferase) expression was seen. HEK293T.17 cells that stably express Renilla Luciferase (for normalization purposes) were plated at 7.5K cells per well in a 96 well plate. The next day, the cells were infected with ZF-CEM technology virus at either 2K or 20K vg/cell. After 24 hours, the cells were treated with varying concentrations of CEM87. After an additional 48 hours (72 hours post initial AAV infection), a Promega Dual Luciferase Assay was run to determine the levels of luciferase.

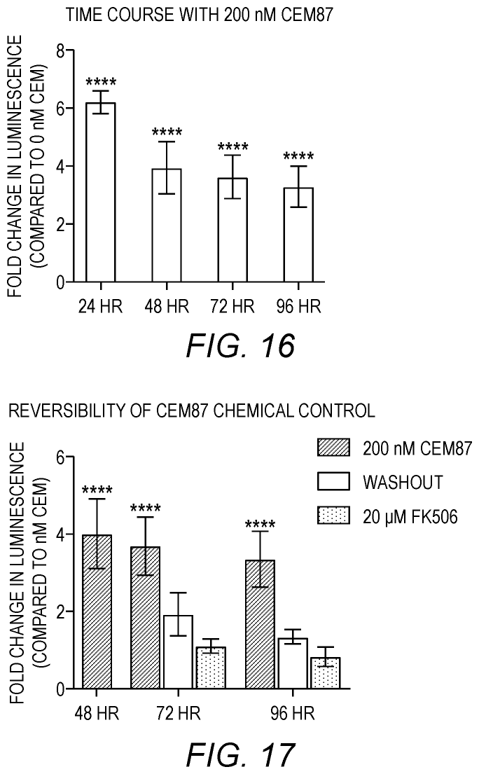

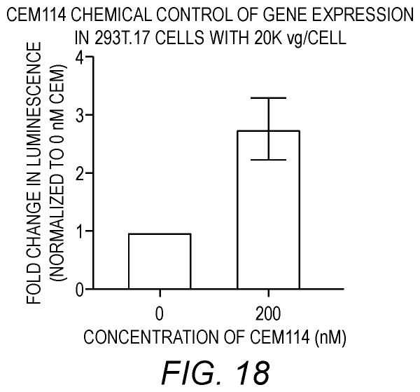

At 20K viral genomes per cell, ZF-CEM technology was able to increase the levels of transgene (firefly luciferase) expression in a statistically significant manner as early as 24 hours and continue to have effects up to 96 hours post CEM87 addition (FIG. 16). The highest fold change in luciferase expression was seen 24 hours after CEM87 dosage (48 hours after AAV infection). HEK293T.17 cells that stably express Renilla Luciferase (for normalization purposes) were plated at 7.5K cells per well in a 96 well plate. The next day, the cells were infected with ZF-CEM technology virus at 20K vg/cell. After 24 hours, the cells were treated with 200 nM CEM87. A Promega Dual Luciferase Assay was run every 24 hours post CEM87 dosage to determine the levels of luciferase over time.

At 20K viral genomes per cell, the impact of ZF-CEM technology on transgene expression was reversible through washout or washout with the addition of FK506, a chemical capable of competing off CEM87 from the transgene it is recruited to (FIG. 17). Reversibility was achieved within 24 hours of removal of CEM87 through washout or washout and treatment with 100× more FK506 (20 mM FK506). HEK293T.17 cells that stably express Renilla Luciferase (for normalization purposes) were plated at 7.5K cells per well in a 96 well plate. The next day, the cells were infected with ZF-CEM technology virus at 20K vg/cell. After 24 hours, the cells were treated with 200 nM CEM87. After 48 hours, wells were either washed out with fresh media or washed out with fresh media that contained 20 mM FK506. A Promega Dual Luciferase Assay was run every 24 hours post washout/FK506 dosage to determine the levels of luciferase over time.

At 20K viral genomes per cell, CEM114 can be used in conjunction with ZF-CEM technology to increase AAV transgene expression. HEK293T.17 cells that stably express Renilla Luciferase (for normalization purposes) were plated at 7.5K cells per well in a 96 well plate. The next day, the cells were infected with ZF-CEM technology virus at 20K vg/cell. After 24 hours, the cells were treated with 200 nM CEM114. After 48 hours, a Promega Dual Luciferase Assay was run to determine the levels of luciferase.

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. (canceled)

2. A method of modulating expression of a transgene from a transgene delivery vector in a subject, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a chemical epigenetic modifier; and
administering to the subject the chemical epigenetic modifier; thereby modulating expression of the transgene.

3. A method of treating a disorder that is treatable by expression of a transgene from a transgene delivery vector in a subject in need thereof, the method comprising:
administering to the subject a transgene delivery vector comprising a polynucleotide comprising a transgene expression cassette and a nucleic acid binding domain recognition sequence;
administering to the subject a fusion protein comprising a nucleic acid binding domain that binds to the recognition sequence fused to a domain that binds a chemical epigenetic modifier; and
administering to the subject the chemical epigenetic modifier;
thereby treating the disorder.

4. The method of claim 2, wherein the transgene delivery vector is a viral vector or a non-viral vector.

5-8. (canceled)

9. The method of claim 2, wherein the transgene encodes a protein or a functional nucleic acid.

10. (canceled)

11. The method of claim 2, wherein the polynucleotide further comprises a sequence encoding the fusion protein.

12. The method of claim 11, wherein the transgene expression cassette and the sequence encoding the fusion protein are operably linked to the same promoter or separate promoters.

13. (canceled)

14. The method of claim 2, wherein the nucleic acid binding domain is a DNA binding domain, optionally wherein the DNA binding domain comprises one of a zinc finger DNA binding domain, a helix-loop-helix DNA binding domain, a bZIP DNA binding domain, an HMG-box DNA binding domain, a transcription activator-like effector DNA binding domain, a transcription factor DNA binding domain, or a restriction endonuclease DNA binding domain; optionally wherein the DNA binding domain is from GAL4, LexA, GCN4, THY1, SYN1, NSE/RU5′, AGRP, CALB2, CAMK2A, CCK, CHAT, DLX6A, EMX1, Cas9, Cas3, Cas4, Cas5, Cas5e (or CasD), Cash, Cas6e, Cas6f, Cas7, Cas8a1, Cas8a2, Cas8b, Cas8c, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu196, or TALES.

15-16. (canceled)

17. The method of claim 2, wherein the nucleic acid binding domain is a RNA binding domain, optionally wherein the RNA binding domain binds a MS2, PP7, GA, or Qβ hairpin motif.

18. (canceled)

19. The method of claim 2, wherein the domain that binds a chemical epigenetic modifier is FK506 binding protein and the chemical epigenetic modifier comprises FK506.

20. The method of claim 2, wherein contacting the transgene delivery vector with the chemical epigenetic modifier or administering the chemical epigenetic modifier to the subject increases expression of the transgene.

21. The method of claim 2, wherein the chemical epigenetic modifier binds to a transcriptional activator protein or complex, optionally BRD4 or CBP/p300, optionally wherein the chemical epigenetic modifier comprises compound 1 (CEM87); 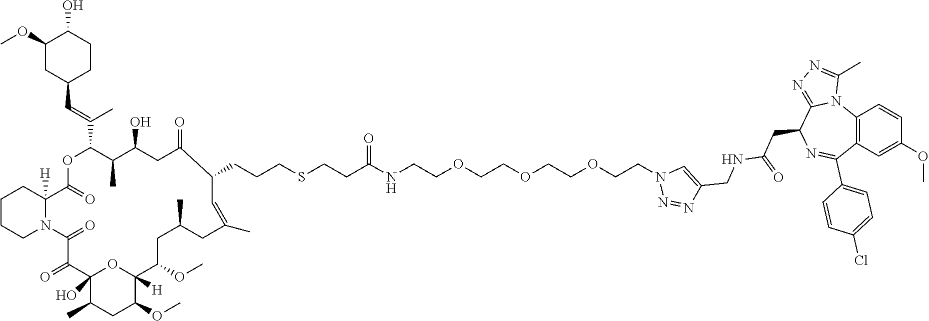
or a pharmaceutically acceptable salt thereof, or
wherein the chemical epigenetic modifier comprises compound 2 (CEM114); 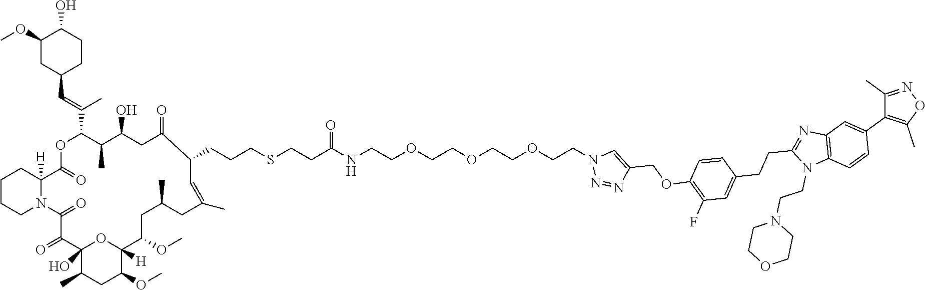 [SMILES: CO[C@H]1C[C@@H](C)C/C(C)=C/[C@@H](CCCSCCC(=O)NCCOCCOCCOCCn2cc(COc3ccc(CCc4nc5cc(-c6c(C)noc6C)ccc5n4CCN4CCOCC4)cc3F)nn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]
or a pharmaceutically acceptable salt thereof.

22-24. (canceled)

25. The method of claim 20, further comprising a step of blocking the increased expression of the transgene by contacting the transgene delivery vector with or administering to the subject an agent that inhibits binding of the fusion protein to the chemical epigenetic modifier.

26. The method of claim 25, wherein the fusion protein comprises FKBP and the agent is FK506.

27. The method of claim 20, further comprising a step of blocking the increased expression of the transgene by stopping the contacting the transgene delivery vector with or the administering to the subject the chemical epigenetic modifier.

28. The method of claim 2, wherein contacting the transgene delivery vector with the chemical epigenetic modifier or administering the chemical epigenetic modifier to the subject decreases expression of the transgene.

29. The method claim 2, wherein the chemical epigenetic modifier binds to a transcriptional inhibitor protein or complex, optionally wherein the chemical epigenetic modifier binds to a histone deacetylase, optionally wherein the chemical epigenetic modifier comprises compound 3 (CEM23): 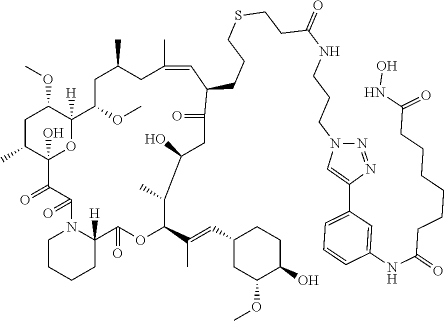 [SMILES: CO[C@H]1C[C@@H](C)C/C(C)=C\[C@@H](CCCSCCC(=O)NCCCn2cc(-c3cccc(NC(=O)CCCCCCC(=O)NO)c3)nn2)C(=O)C[C@H](O)[C@@H](C)[C@@H](/C(C)=C/[C@@H]2CC[C@@H](O)[C@H](OC)C2)OC(=O)[C@@H]2CCCCN2C(=O)C(=O)[C@]2(O)O[C@H]1[C@@H](OC)C[C@H]2C]
or a pharmaceutically acceptable salt thereof.

30-31. (canceled)

32. A transgene delivery vector comprising a polynucleotide comprising a nucleic acid binding domain recognition sequence and a transgene expression cassette comprising a transgene.

33-48. (canceled)

49. A cell comprising the transgene delivery vector of claim 32.

50. A pharmaceutical composition comprising the transgene delivery vector of claim 32.

51. A kit comprising the transgene delivery vector of claim 32.
